(19) United States
(12) Patent Application Publication
(10) Pub. No.: US 2010/0010393 A1
(43) Pub. Date: Jan. 14, 2010

(54) Treatment of Occlusions by External High Intensity Focused Ultrasound

(75) Inventors: Niall Duffy, Tuam (IE); Richard Houben, Lanaken (BE); Ashish Varma, Kinvara (IE); Sean Whelan, Ballybrit (IE)

Correspondence Address: MEDTRONIC VASCULAR, INC.;IP LEGAL DEPARTMENT, 3576 UNOCAL PLACE, SANTA ROSA, CA, 95403, US

(73) Assignee: MEDTRONIC VASCULAR, INC., Santa Rosa, CA (US)

(21) Appl. No.: 12/169,448

(22) Filed: Jul. 8, 2008

(51) Int. Cl. A61N 7/00

(52) U.S. Cl. 601/2

(57) ABSTRACT

An apparatus for treating an occlusion in a vessel inside of a patient. The apparatus includes an external high intensity focused ultrasound transducer configured to be positioned outside of the vessel and to emit ultrasonic waves of energy to the occlusion and to detect ultrasonic waves of energy, an internal ultrasound transducer configured to be positioned inside of the vessel at a position adjacent to or inside the occlusion and to emit ultrasonic waves of energy for detection by the external high intensity focused ultrasound transducer, and a controller configured to control the ultrasonic waves of energy emitted by the external high intensity focused ultrasound transducer based on an electrocardiogram of the patient.

### BACKGROUND OF THE INVENTION

1. Field of the Invention

The present invention is generally related to an apparatus and method to treat occlusions in a vessel, such as a coronary artery, by external high intensity focused ultrasound.

2. Background of the Invention

Stenotic lesions may comprise a hard, calcified substance and/or a softer thrombus material, each of which forms on the lumen walls of a blood vessel and restricts blood flow there through. Intra-luminal treatments such as balloon angioplasty (PTA, PTCA, etc.), stent deployment, atherectomy, and thrombectomy are well known and have proven effective in the treatment of such stenotic lesions. These treatments often involve the insertion of a therapy catheter into a patient's vasculature, which may be tortuous and may have numerous stenoses of varying degrees throughout its length. In order to place the distal end of a catheter at the treatment site, a guidewire is typically introduced and tracked from an incision, through the vasculature, and across the lesion. Then, a catheter (e.g. a balloon catheter), perhaps containing a stent at its distal end, can be tracked over the guidewire to the treatment site. Ordinarily, the distal end of the guidewire is quite flexible so that it can be rotatably steered and pushed through the bifurcations and turns of the typically irregular passageway without damaging the vessel walls.

In some instances, the extent of occlusion of the lumen is so severe that the lumen is completely or nearly completely obstructed, which may be described as a total occlusion. If this occlusion persists for a long period of time, the lesion is referred to as a chronic total occlusion or CTO. Furthermore, in the case of diseased blood vessels, the lining of the vessels may be characterized by the prevalence of atheromatous plaque, which may form total occlusions. The extensive plaque formation of a chronic total occlusion typically has a fibrous cap surrounding softer plaque material. This fibrous cap may present a surface that is difficult to penetrate with a conventional guidewire, and the typically flexible distal tip of the guidewire may be unable to cross the lesion.

Thus, for treatment of total occlusions, stiffer guidewires have been employed to recanalize through the total occlusion. However, due to the fibrous cap of the total occlusion, a stiffer guidewire still may not be able to cross the occlusion. Further, when using a stiffer guidewire, great care must be taken to avoid perforation of the vessel wall.

Further, in a CTO, there may be a distortion of the regular vascular architecture such that there may be multiple small non-functional channels throughout the occlusion rather than one central lumen for recanalization. Thus, the conventional approach of looking for the single channel in the center of the occlusion may account for many of the failures. Furthermore, these spontaneously recanalized channels may be responsible for failures due to their dead-end pathways and misdirecting of the guidewires. Once a “false” tract is created by a guidewire, subsequent attempts with different guidewires may continue to follow the same incorrect path, and it is very difficult to steer subsequent guidewires away from the false tract.

Another equally important failure mode, even after a guidewire successfully crosses a chronic total occlusion, is the inability to advance a balloon or other angioplasty equipment over the guidewire due to the fibrocalcific composition of the chronic total occlusion, mainly both at the “entry” point and at the “exit” segment of the chronic total occlusion. Even with balloon inflations throughout the occlusion, many times there is no antegrade flow of contrast injected, possibly due to the recoil or insufficient channel creation throughout the occlusion.

Atherosclerotic plaques vary considerably in their composition from site to site, but certain features are common to all of them. They contain many cells; mostly these are derived from cells of the wall that have divided wildly and have grown into the surface layer of the blood vessel, creating a mass lesion. Plaques also contain cholesterol and cholesterol esters, commonly referred to as fat. This lies freely in the space between the cells and in the cells themselves. A large amount of collagen is present in the plaques, particularly advanced plaques of the type which cause clinical problems. Additionally, human plaques contain calcium to varying degrees, hemorrhagic material including clot and grumous material composed of dead cells, fat and other debris. Relatively large amounts of water are also present, as is typical of all tissue.

Successful recanalization of chronic total occlusions remains an area where improvements are needed. Approximately 30% of all coronary angiograms in patients with coronary artery disease will show a CTO and its presence often excludes patients from treatment by percutaneous coronary intervention. Acute success rates vary according to the duration of occlusion, the morphology of the lesion and the coronary anatomy, the experience of the operator, the degree of persistence employed, and the type of equipment used. Recanalization rates range between 45-80%, with the highest success in short, recently occluded (<1 month), non-calcified lesions.

Diagnostic Angiography indicates 20-30% of the coronary lesions are fully (100%) occluded. As a result, 10-15% of interventions are chronic total occlusions (CTO). The current success rate of a normal percutaneous transluminal coronary angioplasy (PTCA) procedure for opening a chronic totally occluded coronary artery is below 50%. In attempted cases over longer time (mostly >2 hrs) by highly experienced physicians, the success rate may be higher.

It is desirable to be able to break down the plaque of the occlusion by external methods so that a guidewire may be able to cross the occlusion and a suitable method of treatment may be applied.

### SUMMARY OF THE INVENTION

The present invention describes an apparatus and method to treat lesions in vessels, such as coronary artery occlusions, with external high intensity focused ultrasound.

Embodiments of the present invention provide an apparatus and method to achieve a break down of plaque in a lesion by an external (non-invasive) application of high intensity ultrasound focused on the lesion from an external high intensity focused ultrasound (“HIFU”) transducer. A catheter mounted ultrasound transducer may be positioned adjacent to or within the region of interest and act as a beacon that emits a low energy ultrasound signal. The emitted ultrasound signal may be received by the HIFU transducer to indicate where the high intensity ultrasound should be focused, and together with an electrocardiogram (“ECG”) signal that indicates the end-diastolic (or other defined phase of the heart), adequate alignment and timing of the HIFU signal towards the region of interest in the lesion may be achieved. The HIFU ultrasound signal may result in a softening of the plaque, or even the destruction of, for example, fibrine bonds of the plaque, thereby resulting in at least a partial opening of the vessel.

According to an aspect of the present invention, there is provided an apparatus for treating an occlusion in a vessel inside of a patient. The apparatus includes an external high intensity focused ultrasound transducer configured to be positioned outside of the vessel and to emit ultrasonic waves of energy to the occlusion and to detect ultrasonic waves of energy. The apparatus also includes an internal ultrasound transducer configured to be positioned inside of the vessel at a position adjacent to or inside the occlusion and to emit ultrasonic waves of energy for detection by the external high intensity focused ultrasound transducer. The apparatus further includes a controller configured to control the ultrasonic waves of energy emitted by the external high intensity focused ultrasound transducer based on an electrocardiogram of the patient.

According to an aspect of the invention, there is provided a method for treating an occlusion in a vessel. The method includes positioning an internal ultrasound transducer within the vessel adjacent to or in the occlusion, emitting low power ultrasonic waves of energy with the internal ultrasound transducer, and receiving the low power ultrasonic waves of energy with an external high intensity focused ultrasound transducer positioned on an external surface of a patient. The method also includes generating an electrocardiogram of the patient, and generating pulsed high intensity focused ultrasonic waves of energy towards the occlusion based on the electrocardiogram of the patient.

In an embodiment, the method may also include emitting ultrasonic waves of energy with the external high intensity focused ultrasound transducer, receiving the ultrasonic waves of energy emitted by the external high intensity focused ultrasound transducer with the internal ultrasound transducer, and repositioning the external high intensity focused ultrasound transducer on the external surface based on a strength of signal generated in the internal ultrasound transducer by the receiving of the ultrasonic waves of energy emitted by the external high intensity focused ultrasound transducer.

### BRIEF DESCRIPTION OF THE DRAWINGS

Embodiments of the invention will now be described, by way of example only, with reference to the accompanying schematic drawings in which corresponding reference symbols indicate corresponding parts, and in which:

FIG. 1 is a schematic diagram of a vessel with a chronic total occlusion (“CTO”);

FIG. 2 is a schematic diagram of an embodiment of an apparatus for treating a CTO by external high intensity focused ultrasound;

FIG. 3 is a schematic diagram of an embodiment of a distal end of an elongated member of the apparatus of FIG. 2;

FIG. 4 is a schematic diagram of the elongated member in the vessel of FIG. 1;

FIG. 5 is a schematic diagram of the elongated member in the CTO and an external high intensity focused ultrasound (“HIFU”) transducer of the apparatus of FIG. 2; and

FIG. 6 is a schematic diagram of the external HIFU transducer of FIG. 5 treating the CTO.

### DETAILED DESCRIPTION OF THE INVENTION

The following detailed description is merely exemplary in nature and is not intended to limit the invention or the application and use of the invention. Furthermore, there is no intention to be bound by any expressed or implied theory presented in the preceding technical field, background, brief summary or the following detailed description.

Specific embodiments of the present invention are now described with reference to the figures, wherein like reference numbers indicate identical or functionally similar elements. The terms “distal” and “proximal” are used in the following description with respect to a position or direction relative to the treating clinician. “Distal” or “distally” are a position distant from or in a direction away from the clinician. “Proximal” and “proximally” are a position near or in a direction toward the clinician.

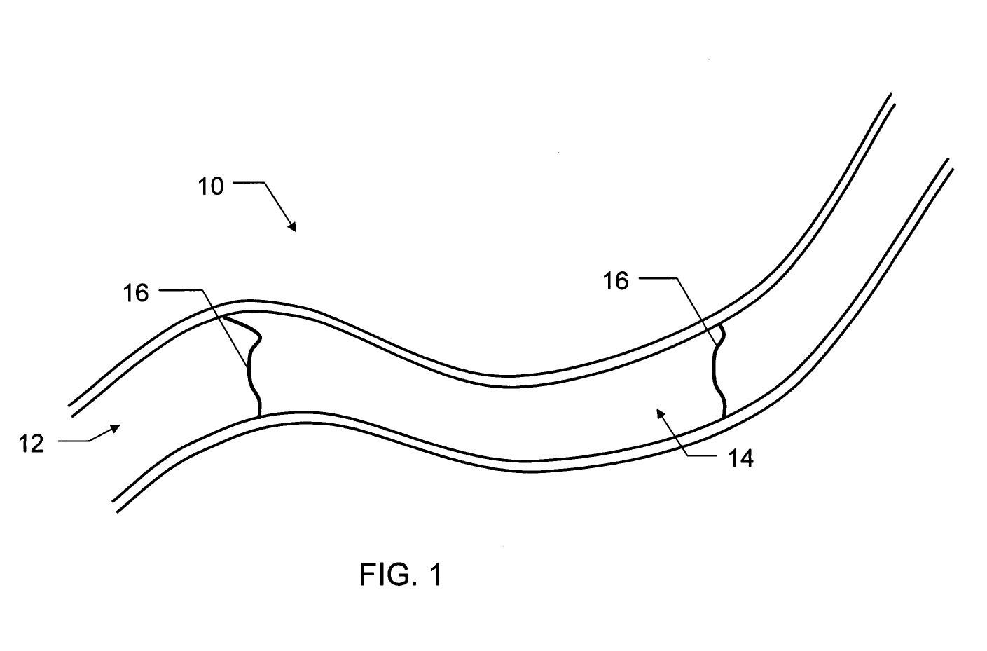

FIG. 1 illustrates a vessel 10 defining an inner lumen 12 through which blood flows. The vessel 10 may be a coronary artery, although embodiments of the invention are not limited to the treatment of coronary arteries. A chronic total occlusion (CTO) 14, which includes plaque, is located within the lumen 12. The CTO 14 has an end cap 16 located at each end thereof. The end caps 16 may include plaque that is relatively harder than the plaque within the remaining portion of the CTO 14. Although a CTO is illustrated, embodiments of the invention are not limited to treating CTO's and may be used to treat other lesions in vessels that are less than totally occluded.

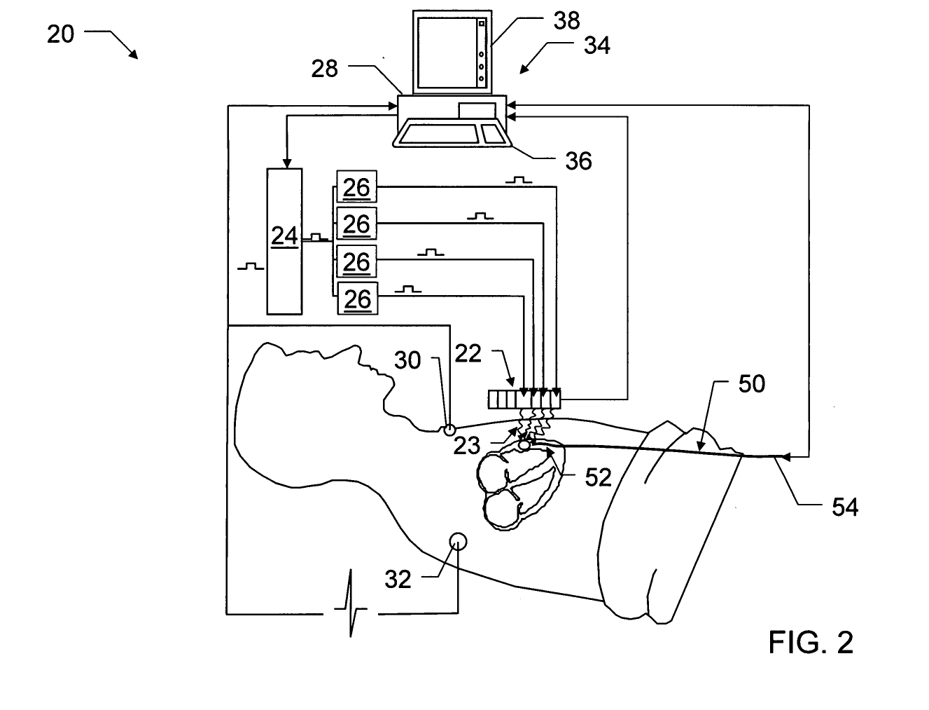

FIG. 2 illustrates an apparatus 20 configured to treat the CTO 14 according to embodiments of the invention. As illustrated, the apparatus 20 includes at least one high intensity focused ultrasound (“HIFU”) transducer 22 that is configured to be positioned outside the body of the patient, such as on the patient's chest, as illustrated in FIG. 2. The transducer 22 is operatively connected to a generator 24 that is configured to generate signals that energize the HIFU transducer 22 to emit pulses of high intensity focused ultrasonic waves 23 to the CTO 14. A plurality of delay units 26 may also be connected to the generator 24. The delay units 26 may be configured to delay the pulses being transmitted to the transducer 22 so that the timing of the ultrasonic waves 23 emitted by the HIFU transducer 22 may be controlled.

The generator 24 is also connected to or in communication with a controller 28. The controller 28 may be configured to control the generator 24 and the delay units 26 so that the intensity, frequency, and the duration of the ultrasonic waves 23 may be coordinated and optimized for heating and ablating the plaque within the CTO 14, without causing damage to the surrounding tissue. This may allow for the heat being generated within the patient to be kept to a minimum, while still being able to ablate the plaque in the CTO 14. For example, it is desirable to avoid production of heat above about 41° C. by operating the apparatus 20 at a low duty cycle.

As illustrated in FIG. 2, the controller 28 is also configured to receive signals representative of the patient's electrocardiogram (“ECG”), and control the generator 24 and the delay units 26, and therefore the HIFU transducer 22, based on the electrocardiogram. To create the ECG, a pair of electrodes, including a first electrode 30 and a second electrode 32 may be attached to the patient's skin at spaced apart locations so that the voltage between the electrodes 30, 32 may be measured. As is known in the art, the voltage that is measured over time provides information on the heart, such as the patient's heart beat, etc. The use of the ECG to determine when to send signals to the generator 24 and the delay units 26 is discussed in further detail below.

The apparatus 20 may also include a user interface 34 that may include an input device 36 that allows the clinician to turn the apparatus 20 on and off, adjust settings of the apparatus 20, trigger the generator 24 and delay units 26 to provide the signal to the HIFU transducer 22, etc. The input device 36 may include buttons, switches, knobs, or any other suitable devices that allow the clinician to change an operating condition of the apparatus 20. The user interface 34 may also include an output device 38, such as a video monitor, that is configured to provide the clinician with information about the apparatus 20 and the CTO 14. For example, an image of the vessel 10 and the CTO 14 may be displayed by the output device 38 by using known imaging techniques, such as fluoroscopy.

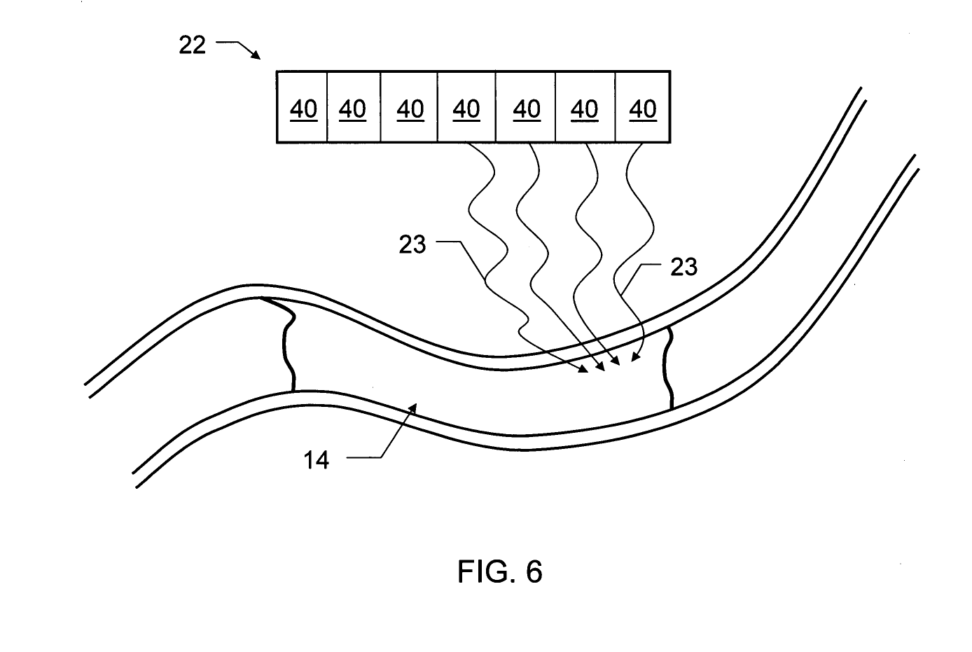

The HIFU transducer 22 may include a plurality of ultrasound transducers 40 that can be positioned on the patient's chest. The plurality of ultrasound transducers 40 may be individually controllable so that the position and orientation, i.e. focal point, of each transducer 40 may be set independent from each other. By adequate focusing and timing of signals to the transducers 40, the ultrasonic waves 23 emitted by the HIFU transducer 22 may be focused on a desired location of the CTO 14, as shown in FIG. 6.

Specifically, in an embodiment, the transducers 40 may be used to create an adequate ultrasound power density that is concentrated within a region of interest for a suitable duration so that an ultrasound impulse is created. The ultrasonic waves 23, which may together be called an ultrasound impulse, are configured to produce enough heat and mechanical forces to break down plaque in the CTO 14. The transducers 40 are also configured so that the ultrasound impulse decays rapidly to avoid damage to the surrounding tissue. In order to minimize the amount of heat that is produced by the ultrasound impulse, the apparatus 20 should be operated at a low duty cycle.

Each transducer 40 may have any suitable construction that can provide the desired ultrasonic waves 23. For example, each transducer 40 may include a single piezoelectric crystal, or each transducer may include an array of piezoelectric crystals. The piezoelectric crystal is configured to oscillate at a high frequency when voltage is provided by the generator 24 and delay units 26, thereby causing the transducer 40 to emit the ultrasonic wave 23. The piezoelectric crystal may comprise lead zirconate titanate, or any other suitable material. In an embodiment one or more of the transducers may include a plurality of piezoelectric micromachined ultrasound transducers.

As shown in FIG. 2, the apparatus 20 may include an elongated member 50, such as a catheter or a guidewire that is configured to enter the lumen 12 and be advanced to the CTO 14. The elongated member 50 has a distal end 52 and a proximal end 54. The distal end 52 may be inserted into the lumen 12, while the proximal end 54 remains outside of the patient so that the clinician may maneuver the elongated member 50. As illustrated in FIG. 2, the proximal end 54 of the elongated member 50 is connected to, or in communication with, the controller 28.

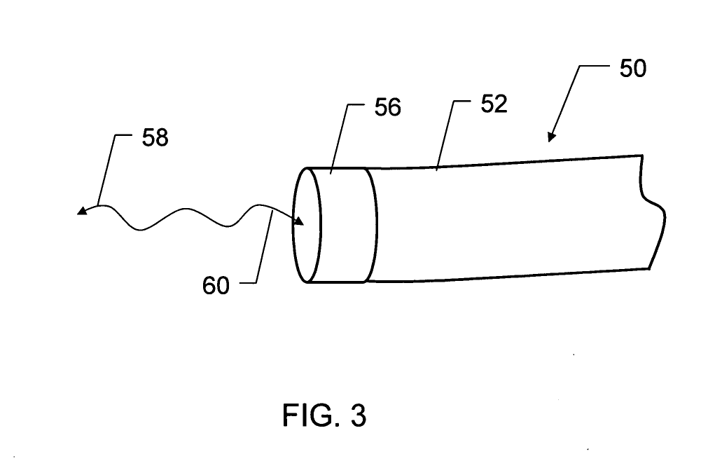

The elongated member 50 may include an ultrasound transducer 56 at the distal end 52, as shown in more detail in FIG. 3. The controller 28 is configured to provide a signal to the ultrasound transducer 56 so that the transducer 56 will emit an ultrasonic signal 58. The ultrasound transducer 56 may be a single element ultrasound transducer and may have a concave shape that is configured to allow the ultrasonic signal 58 that is provided by the ultrasound transducer 56 to be focused at a predefined focal depth.

The ultrasound transducer 56 may include a piezoelectric crystal that is configured to oscillate at a high frequency when voltage is provided by the controller 28, thereby emitting the ultrasonic signal 58. The piezoelectric crystal may comprise lead zirconate titanate, or any other suitable material. The ultrasound transducer 56 may include a plurality of piezoelectric crystals that may be arranged in an annular array. In an embodiment, the ultrasound transducer 56 may include a plurality of piezoelectric micromachined ultrasound transducers.

The controller 28 is also configured to receive a signal from the transducer 56 and convert the signal to information about the location of the CTO 14, or about the ultrasonic waves 23 emitted by the HIFU transducer 22. For example, as discussed in further detail below, the ultrasound transducer 56 may act as a beacon to guide the ultrasonic waves 23 from the HIFU transducer 22 to the target location, i.e., the CTO 14.

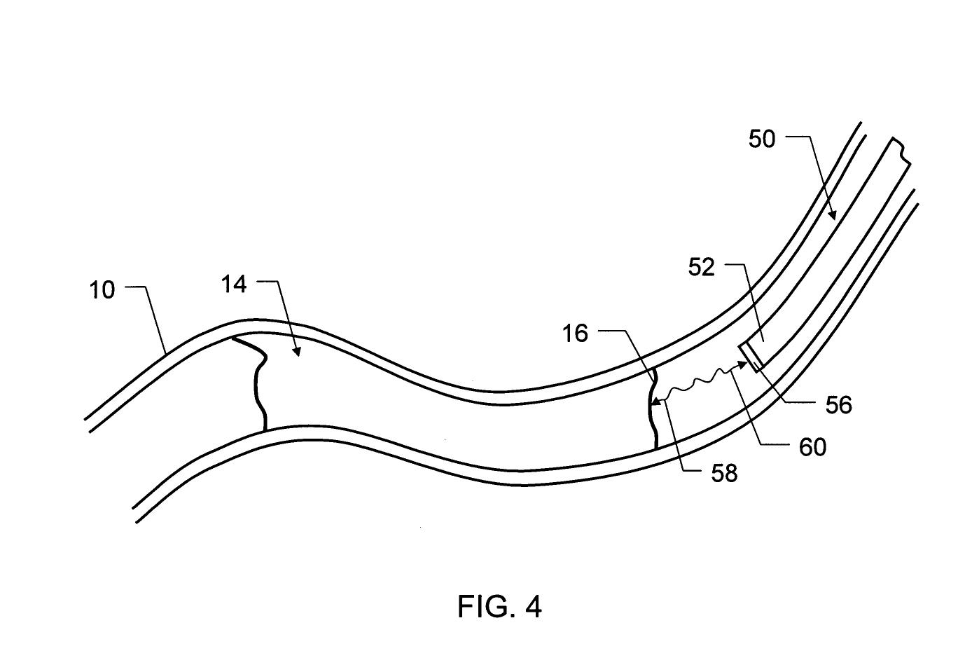

In an embodiment, the ultrasound transducer 56 may be configured to emit the ultrasonic signal 58 towards the CTO 14 and to receive a reflected signal 60 from the CTO 14, as shown in FIG. 4. Such a configuration may be used to locate the CTO 14 within the lumen 12. Specifically, the controller 28 may be configured to determine the length of time it takes for the emitted signal 58 to leave the ultrasound transducer 56, reflect off of the CTO 14, and be received by the ultrasound transducer 56 in the form of the reflected signal 60. The controller 28 may then correlate the length of time into a distance, as is known in the art.

Once the distal end 52 of the elongated member 50 is located adjacent to the CTO 14, the clinician may be able to push the distal end 52 of the elongated member 50 through the end cap 16 and into the CTO 14, if the end cap 16 of the CTO 14 is sufficiently soft. In an embodiment, the ultrasound transducer 56 may also be configured to emit ultrasound impulses that are suitable to ablate the end cap 16 of the CTO 14 so that the distal end 52 of the elongated member 50 may be inserted into the CTO 14.

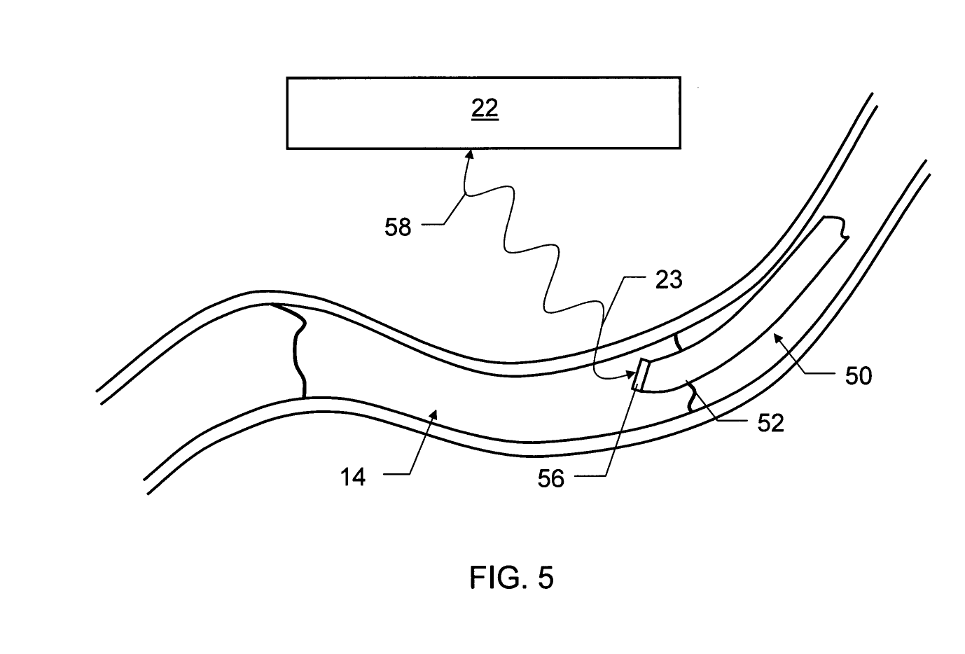

Once the CTO 14 has been located and the distal end 52 of the elongated member 50 has been positioned adjacent to the CTO 14, or if possible, within the CTO 14, the controller 28 may send a signal to the ultrasound transducer 56 to emit low power (diagnostic level) ultrasonic signals 58, or pulses, that may be received by the external HIFU transducer 22, as shown in FIG. 5. This allows the clinician to complete what may be called a first stage alignment. Specifically, the clinician may move the HIFU transducer 22 on the patient's chest until the ultrasonic signals 58 being emitted by the ultrasound transducer 56 are detected by the HIFU transducer 22. In this way, the ultrasound transducer 56 may act as a so-called “beacon” to the HIFU transducer 22.

Next, the external HIFU transducer 22 can be aligned more accurately in a second stage alignment by emitting ultrasonic pulses 23 towards the ultrasound transducer 56 of the elongated member 50 so that the ultrasound transducer 56 may sense the ultrasound pulses 23 being emitted by the external HIFU transducer 22. The external HIFU transducer 22 may be repositioned until optimal focus is achieved. Optimal focus may be achieved when maximum power is received by the ultrasound transducer 56, as determined by the controller 28.

After the HIFU transducer 22 has been positioned where optimal focus on the CTO 14 is achieved, the controller 28 may signal the generator 24 and delay units 26 so that the HIFU transducer 22 may emit controlled impulses of ultrasonic waves 23 to the CTO 14. The ultrasound impulses are controlled by the controller 28 in terms of intensity and frequency, at a low duty cycle, to achieve destruction of fibrine bonds of the plaque in the CTO 14. Depending on the hardness of the plaque in the CTO 14 and the size of the occlusion, at least a partial opening of the vessel 10 may be achieved. For softer plaque and occlusions, the apparatus 20 may be used to substantially or even completely remove the CTO 14. After the vessel 10 has been at least partially opened, the clinician may attempt to cross the CTO 14 with a guidewire and/or catheter so that the CTO 14 may be further treated by known methods.

The use of the ECG may provide information to the controller 28 of the exact timing for the HIFU transducer 22 to apply a short high intensity ultrasonic impulse that is focused on the CTO 14. In an embodiment, the impulse may be applied at the phase of the heart where the position of the CTO 14 is stable for the longest period in time, such as at the end-diastolic, or other defined phase of the heart. Applying the high intensity ultrasonic impulse during a phase of the heart where the position of the CTO 14 is stable may increase the chance that the impulse has the desired effect. As a result, fewer impulses may be needed, which may minimize any adverse side effects.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient roadmap for implementing an exemplary embodiment of the invention, it being understood that various changes may be made in the function and arrangement of elements described in an exemplary embodiment without departing from the scope of the invention as set forth in the appended claims.

What is claimed is:

1. An apparatus for treating an occlusion in a vessel inside of a patient, the apparatus comprising:
an external high intensity focused ultrasound transducer configured to be positioned outside of the vessel and to emit ultrasonic waves of energy to the occlusion and to detect ultrasonic waves of energy;
an internal ultrasound transducer configured to be positioned inside of the vessel at a position adjacent to or inside the occlusion and to emit ultrasonic waves of energy for detection by the external high intensity focused ultrasound transducer; and
a controller configured to control the ultrasonic waves of energy emitted by the external high intensity focused ultrasound transducer based on an electrocardiogram of the patient.

2. An apparatus according to claim 1, further comprising a pair of electrodes constructed and arranged to generate the electrocardiogram.

3. An apparatus according to claim 1, further comprising a elongated member comprising the internal ultrasound transducer at a distal end thereof, the elongated member being configured to enter the vessel and locate the internal ultrasound transducer to the position adjacent to or inside the occlusion.

4. An apparatus according to claim 3, wherein the elongated member is a catheter.

5. An apparatus according to claim 1, wherein the internal ultrasound transducer is configured to receive the ultrasonic waves of energy emitted by the external high intensity focused ultrasound transducer and to communicate a signal based on the received ultrasonic waves to the controller, and wherein the controller is configured to process the signal and provide an indication of whether the external high intensity focused ultrasound transducer is optimally positioned.

6. An apparatus according to claim 1, further comprising a generator configured to generate signals to the external high intensity focused ultrasound transducer so that the ultrasonic waves of energy are pulsed to the occlusion on a low duty cycle.

7. An apparatus according to claim 6, further comprising a plurality of delay units constructed and arranged to received the signals generated by the generator and to delay the signals being provided to the external high intensity focused ultrasound transducer to create the pulses.

8. An apparatus according to claim 1, wherein the internal ultrasound transducer is constructed and arranged to emit high intensity ultrasonic waves of energy towards the occlusion to ablate an end cap of the occlusion.

9. A method for treating an occlusion in a vessel, the method comprising:
positioning an internal ultrasound transducer within the vessel adjacent to or in the occlusion;
emitting low power ultrasonic waves of energy with the internal ultrasound transducer;
receiving the low power ultrasonic waves of energy with an external high intensity focused ultrasound transducer positioned on an external surface of a patient;
generating an electrocardiogram of the patient; and
generating pulsed high intensity focused ultrasonic waves of energy towards the occlusion based on the electrocardiogram of the patient.

10. A method according to claim 9, further comprising:
emitting ultrasonic waves of energy with the external high intensity focused ultrasound transducer;
receiving the ultrasonic waves of energy emitted by the external high intensity focused ultrasound transducer with the internal ultrasound transducer; and
repositioning the external high intensity focused ultrasound transducer on the external surface based on a strength of signal generated in the internal ultrasound transducer by the receiving of the ultrasonic waves of energy emitted by the external high intensity focused ultrasound transducer.

11. A method according to claim 9, wherein the pulsed high intensity focused ultrasonic waves are generated at an end of a heart beat.

12. A method according to claim 9, further comprising tracking an elongated member in the vessel to the occlusion, the catheter comprising the internal ultrasound transducer.

13. A method according to claim 9, wherein the pulsed high intensity focused ultrasonic waves of energy are pulsed towards the occlusion on a low duty cycle.

14. A method according to claim 9, further comprising emitting high intensity focused ultrasonic waves of energy towards the occlusion to ablate an end cap of the occlusion with the internal ultrasound transducer.
